(19) United States
(12) Patent Application Publication
(10) Pub. No.: US 2022/0259209 A1
(43) Pub. Date: Aug. 18, 2022

(54) PROCESS FOR THE PREPARATION OF ABROCITINIB

(71) Applicant: Glenmark Life Sciences Limited, Mumbai (IN)

(72) Inventors: Shekhar Bhaskar Bhirud, Mumbai (IN); Suresh Mahadev Kadam, Mumbai (IN); Sachin Baban Gavhane, Dombivli (East) (IN); Vishweshwar Peddy, Hyderabad (IN); Shailendra Nilkanth Bhadane, Koparkhairane, Navi Mumbai (IN); Vinayak Kacheshwar Bhujade, Kopargaon (IN)

(21) Appl. No.: 17/621,955

(22) PCT Filed: Jun. 16, 2020
(86) PCT No.: PCT/IB2020/055596

§ 371 Date: Dec. 22, 2021

(30) Foreign Application Priority Data
Jun. 27, 2019 (IN) 201921025659

(51) Int. Cl. C07D 487/04; A61K 9/14

(52) U.S. Cl. CPC C07D 487/04; C07B 2200/13; A61K 9/146

(57) ABSTRACT

The present invention relates to crystalline abrocitinib characterized by X-ray powder diffraction (XRPD) spectrum having peak reflections at about 12.9, 14.7, 19.4, 23.2 and 25.2±0.2 degrees 2 theta, and process for its preparation. The present invention relates to amorphous solid dispersion comprising abrocitinib or salt thereof together with at least one pharmaceutically acceptable carrier and process for its preparation.

### PRIORITY

This application claims the benefit of Indian Provisional Application 201921025659 filed on Jun. 27, 2019 entitled “PROCESS FOR PREPARATION OF ABROCITINIB”, the contents of which are incorporated herein by reference.

### FIELD OF THE INVENTION

The present invention relates to crystalline abrocitinib and process for its preparation. The present invention relates to amorphous solid dispersion comprising abrocitinib or salt thereof together with at least one pharmaceutically acceptable carrier and process for its preparation.

### BACKGROUND OF THE INVENTION

Abrocitinib, also known as, N-[cis-3-[methyl(7H-pyrrolo[2,3-d]pyrimidin-4-yl)amino]cyclobutyl]propane-1-sulfonamide, is represented by the structure of formula I.

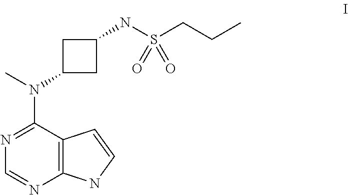 [SMILES: CCCS(=O)(=O)N[C@H]1C[C@@H](N(C)c2ncnc3[nH]ccc23)C1]

Abrocitinib is janus tyrosine kinase (Jak1) inhibitor, indicated for the potential oral treatment of moderate-to-severe atopic dermatitis (AD).

The discovery of polymorphic forms of active pharmaceutical ingredients (“APIs”) provides opportunities to improve the performance characteristics, the solubility, stability, flowability, tractability and compressibility of drug substances and the safety and efficacy of drug products of a pharmaceutical product. Such discoveries enlarge the repertoire of materials that a formulation scientist has available for designing, for example, a pharmaceutical dosage form of a drug with a targeted release profile or other desired characteristic.

The object of the present invention is to provide crystalline abrocitinib, amorphous abrocitinib, and amorphous solid dispersions comprising abrocitinib.

### SUMMARY OF THE INVENTION

The present invention provides a process for the preparation of crystalline abrocitinib characterized by X-ray powder diffraction (XRPD) spectrum having peak reflections at about 12.9, 14.7, 19.4, 23.2 and 25.2±0.2 degrees 2 theta, the process comprising:

(a) dissolving abrocitinib in a solvent selected from the group consisting of ethers, ketones, esters, haloalkanes, amides, alcohols, and mixtures thereof;
(b) obtaining crystalline abrocitinib from the solution of step (a); and
(c) isolating the crystalline abrocitinib.

In another embodiment, the present invention provides an amorphous solid dispersion comprising abrocitinib or salt thereof together with at least one pharmaceutically acceptable carrier.

In another embodiment, the present invention provides a process for the preparation of an amorphous solid dispersion of abrocitinib or salt thereof together with at least one pharmaceutically acceptable carrier, the process comprising:

(a) providing a solution or mixture of abrocitinib or salt thereof together with at least one pharmaceutically acceptable carrier in a solvent; and
(b) obtaining the amorphous solid dispersion of abrocitinib or salt thereof together with at least one pharmaceutically acceptable carrier from the solution or mixture of step (a).

### BRIEF DESCRIPTION OF THE DRAWINGS

FIG. 1 is a characteristic XRPD of crystalline abrocitinib as obtained in Example 8.

FIG. 2 is a DSC thermogram of crystalline abrocitinb as obtained in Example 8.

FIG. 3 is a TGA thermogram of crystalline abrocitinib as obtained in Example 8.

FIG. 4 is a characteristic XRPD of amorphous solid dispersion comprising abrocitinib and hydroxypropyl cellulose as obtained in Example 16.

FIG. 5 is a characteristic XRPD of amorphous solid dispersion comprising abrocitinib and polyvinyl pyrrolidone as obtained in Example 17.

FIG. 6 is a characteristic XRPD of amorphous solid dispersion comprising abrocitinib and polyvinyl pyrrolidone as obtained in Example 18.

FIG. 7 is a characteristic XRPD of amorphous solid dispersion comprising abrocitinib and polyvinyl pyrrolidone as obtained in Example 19.

### DETAILED DESCRIPTION OF THE INVENTION

The present invention provides a crystalline abrocitinib.

In the present application, the term “room temperature” means a temperature of about 25° C. to about 30° C.

In one embodiment, the present invention provides a crystalline abrocitinib characterized by X-ray powder diffraction (XRPD) spectrum having peak reflections at about 12.9, 14.7, 19.4, 23.2 and 25.2±0.2 degrees 2 theta.

In one embodiment, the present invention provides a crystalline abrocitinib characterized by X-ray powder diffraction (XRPD) spectrum having peak reflections at about 12.5, 12.9, 14.7, 17.5, 17.9, 19.4, 20.6, 23.2, 24.9 and 25.2±0.2 degrees 2 theta.

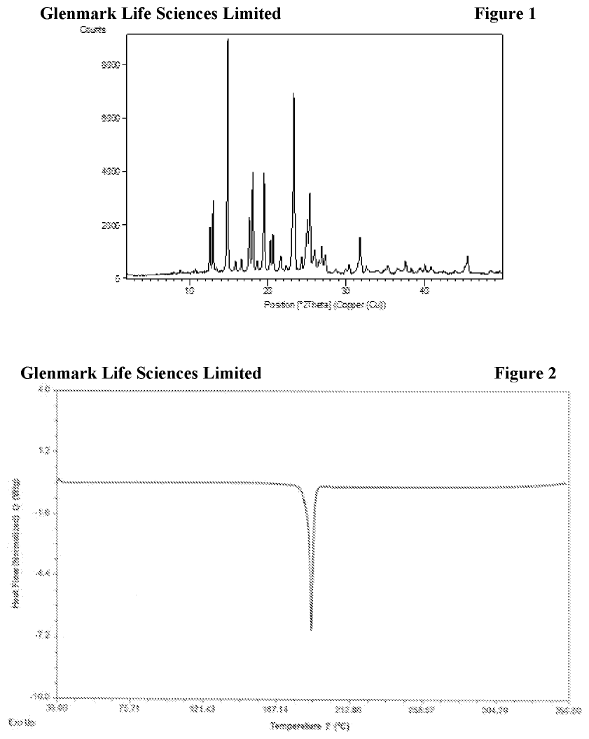

In one embodiment, the present invention provides a crystalline abrocitinib characterized by an X-ray powder diffraction (XRPD) spectrum having peak reflections at about 12.9, 14.7, 19.4, 23.2 and 25.2±0.2 degrees 2 theta which is substantially in accordance with FIG. 1.

In one embodiment, the present invention provides a crystalline abrocitinib characterized by DSC thermogram having an endothermic peak at about 189±2° C.

In one embodiment, the present invention provides a crystalline abrocitinib characterized by DSC thermogram having an endothermic peak at about 189±2° C. which is substantially in accordance with FIG. 2.

In one embodiment, the present invention provides a crystalline abrocitinib characterized by X-ray powder diffraction (XRPD) spectrum having peak reflections at about 12.9, 14.7, 19.4, 23.2 and 25.2±0.2 degrees 2 theta and DSC thermogram having an endothermic peak at about 189±2° C.

In one embodiment, the present invention provides a crystalline abrocitinib characterized by TGA thermogram, showing a weight loss of about 0.07 weight % to 0.4 weight % determined over the temperature range of 25° C. to 150° C. and heating rate 10° C./min.

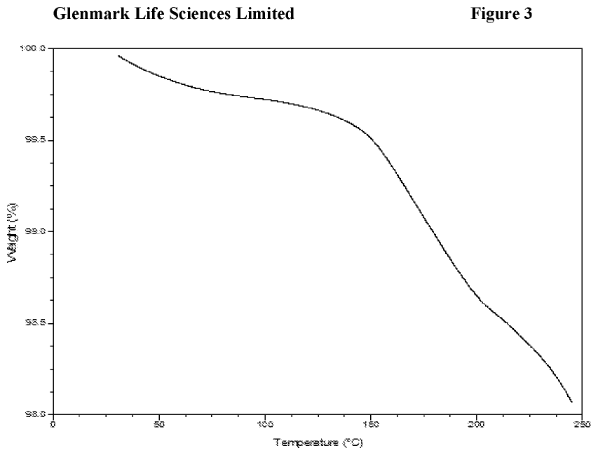

In one embodiment, the present invention provides a crystalline abrocitinib characterized by TGA thermogram, showing a weight loss of about 0.07 weight % to 0.4 weight % determined over the temperature range of 25° C. to 150° C. and heating rate 10° C./min which is substantially in accordance with FIG. 3.

In one embodiment, the present invention provides a crystalline abrocitinib characterized by X-ray powder diffraction (XRPD) spectrum having peak reflections at about 12.9, 14.7, 19.4, 23.2 and 25.2±0.2 degrees 2 theta and TGA thermogram, showing a weight loss of about 0.07 weight % to 0.4 weight % determined over the temperature range of 25° C. to 150° C. and heating rate 10° C./min.

In one embodiment, the present invention provides a crystalline abrocitinib characterized by data selected from the group consisting of: an X-ray powder diffraction (XRPD) pattern as depicted in FIG. 1, a DSC thermogram as depicted in FIG. 2; a TGA thermogram as depicted in FIG. 3; and any combination thereof.

In one embodiment, the present invention provides a process for the preparation of crystalline abrocitinib characterized by X-ray powder diffraction (XRPD) spectrum having peak reflections at about 12.9, 14.7, 19.4, 23.2 and 25.2±0.2 degrees 2 theta, the process comprising:

(a) dissolving abrocitinib in a solvent selected from the group consisting of ethers, ketones, esters, haloalkanes, amides, alcohols, and mixtures thereof;
(b) obtaining crystalline abrocitinib from the solution of step (a); and
(c) isolating the crystalline abrocitinib.

In (a) of the process for the preparation of crystalline abrocitinib, abrocitinib is dissolved in a solvent selected from the group consisting of ethers, ketones, esters, haloalkanes, amides, alcohols, and mixtures thereof.

In one embodiment, the solvent used for dissolution of abrocitinib may be selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof.

In one embodiment, the solvent used for dissolution of abrocitinib may be selected from the group consisting of C₂₋₁₀ ethers such as such as diethyl ether, diisopropyl ether, methyl tert-butyl ether, tetrahydrofuran, dioxane, dimethoxy ethane, 2-methyltetrahydrofuran and the like; C₃₋₁₀ ketones such as acetone, methyl isobutyl ketone, ethyl methyl ketone and the like; C₂₋₁₀ esters such as methyl acetate, ethyl acetate, n-propyl acetate, isopropyl acetate, tert-butyl acetate and the like; C₁₋₆ haloalkanes such as methylene dichloride, ethylene dichloride, chloroform and the like; C₁₋₈ amides such as dimethyl formamide; dimethyl acetamide and the like; C₁₋₆ alcohols such as methanol, ethanol, 1-propanol, 2-propanol, 1-butanol, 2-butanol, 1-pentanol and the like; and mixtures thereof.

Suitable temperature for dissolution of abrocitinib may range from about 20° C. to about reflux temperature of the solvent.

In one embodiment, abrocitinib is dissolved at about room temperature.

In one embodiment, abrocitinib is dissolved at about reflux temperature of the solvent.

In one embodiment, abrocitinib is dissolved in the selected solvent by stirring the mixture of abrocitinib in the selected solvent. Stirring may be continued for any desired time period to achieve a complete dissolution of abrocitinib. The stirring time may range from about 30 minutes to about 10 hours, or longer. The solution may be optionally treated with charcoal and filtered to get a particle-free solution.

In (b) of the process for the preparation of crystalline abrocitinib, crystalline abrocitinib is obtained from the solution of step (a).

In one embodiment, the step (b) of obtaining crystalline abrocitinib comprises:

(i) cooling and stirring the solution obtained in (a); or
(ii) removing the solvent from the solution obtained in (a); or
(iii) treating the solution of step (a) with an anti-solvent to form a mixture and optionally, cooling and stirring the obtained mixture.

In one embodiment, the crystalline abrocitinib is obtained by cooling and stirring the solution of step (a).

In one embodiment, the solution obtained in step (a) is cooled to about 0° C. to about room temperature.

In one embodiment, the stirring time may range from about 30 minutes to about 10 hours, or longer.

In one embodiment, the crystalline abrocitinib is obtained by removing the solvent from the solution obtained in (a). Removal of solvent may be accomplished by substantially complete evaporation of the solvent; or concentrating the solution, cooling the solution if required and filtering the obtained solid. The solution may also be completely evaporated in, for example, a rotavapor, a vacuum paddle dryer or in a conventional reactor under vacuum above about 720 mm Hg.

In one embodiment, the crystalline abrocitinib is obtained by adding an anti-solvent to the solution obtained in (a) to form a mixture and optionally, cooling and stirring the obtained mixture. The stirring time may range from about 30 minutes to about 10 hours, or longer. The temperature may range from about 0° C. to about room temperature.

The anti-solvent is selected such that crystalline abrocitinib is precipitated out from the solution.

The anti-solvent is selected from the group consisting of hydrocarbons, ethers, ketones, esters, haloalkanes, amides, alcohols, water, and mixtures thereof.

In one embodiment, the anti-solvent is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, water, and mixtures thereof.

In one embodiment, the anti-solvent is selected from the group consisting of C₂₋₁₀ ethers such as such as diethyl ether, diisopropyl ether, methyl tert-butyl ether, tetrahydrofuran, dioxane, dimethoxy ethane, 2-methyltetrahydrofuran and the like; C₃₋₁₀ ketones such as acetone, methyl isobutyl ketone, ethyl methyl ketone and the like; C₂₋₁₀ esters such as methyl acetate, ethyl acetate, n-propyl acetate, isopropyl acetate, tert-butyl acetate and the like; C₁₋₆ haloalkanes such as methylene dichloride, ethylene dichloride, chloroform and the like; C₁₋₈ amides such as dimethyl formamide; dimethyl acetamide and the like; C₁₋₆ alcohols such as methanol, ethanol, 1-propanol, 2-propanol, 1-butanol, 2-butanol, 1-pentanol and the like; water; and mixtures thereof.

In (c) of the process for the preparation of crystalline abrocitinib, the crystalline abrocitinib is isolated by any method known in the art. The method, may involve any of techniques, known in the art, including filtration by gravity or by suction, centrifugation, and the like.

In one embodiment, the present invention provides a process for the preparation of crystalline abrocitinib characterized by X-ray powder diffraction (XRPD) spectrum having peak reflections at about 12.9, 14.7, 19.4, 23.2 and 25.2±0.2 degrees 2 theta, the process comprising:

(a) dissolving abrocitinib in a solvent selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof;
(b) obtaining crystalline abrocitinib from the solution of step (a); and
(c) isolating the crystalline abrocitinib.

In one embodiment, the present invention provides a process for the preparation of crystalline abrocitinib, the process comprising:

(a) dissolving abrocitinib in a solvent selected from the group consisting of ethers, ketones, esters, haloalkanes, amides, alcohols, and mixtures thereof;
(b) obtaining crystalline abrocitinib from the solution of step (a) by cooling and stirring the solution obtained in (a); and
(c) isolating the crystalline abrocitinib.

In one embodiment, the solvent used in step (a) is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof.

In one embodiment, the solvent used in step (a) is selected from the group consisting of C₂₋₁₀ ethers such as tetrahydrofuran, C₃₋₁₀ ketones such as acetone, C₂₋₁₀ esters such as ethyl acetate, C₁₋₆ alcohols such as methanol, and mixtures thereof.

In one embodiment, the present invention provides a process for the preparation of crystalline abrocitinib, the process comprising:

(a) dissolving abrocitinib in a solvent selected from the group consisting of C₂₋₁₀ ethers, C₁₋₆ alcohols, and mixtures thereof;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in tetrahydrofuran-methanol mixture;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in a solvent selected from the group consisting of C₃₋₁₀ ketones, C₁₋₆ alcohols, and mixtures thereof;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in acetone-methanol mixture;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in a solvent selected from the group consisting of C₂₋₁₀ esters, C₁₋₆ alcohols, and mixtures thereof;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in ethyl acetate-methanol mixture;
(b) obtaining crystalline abrocitinib from the solution of step (a) by cooling and stirring the solution obtained in (a); and
(c) isolating the crystalline abrocitinib.

In one embodiment, the present invention provides a process for the preparation of crystalline abrocitinib, the process comprising:

(a) dissolving abrocitinib in a solvent selected from the group consisting of ethers, ketones, esters, haloalkanes, amides, alcohols, and mixtures thereof;
(b) obtaining crystalline abrocitinib from the solution of step (a) by removing the solvent from the solution obtained in (a); and
(c) isolating the crystalline abrocitinib.

In one embodiment, the solvent used in step (a) is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof.

In one embodiment, the solvent used in step (a) is selected from the group consisting of C₂₋₁₀ ethers such as tetrahydrofuran, C₃₋₁₀ ketones such as acetone, C₂₋₁₀ esters such as ethyl acetate, C₁₋₆ haloalkanes such as methylene dichloride, C₁₋₆ alcohols such as methanol, and mixtures thereof.

In one embodiment, the present invention provides a process for the preparation of crystalline abrocitinib, the process comprising:

(a) dissolving abrocitinib in a solvent selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof;
(b) obtaining crystalline abrocitinib from the solution of step (a) by removing the solvent from the solution obtained in (a); and
(c) isolating the crystalline abrocitinib.

In one embodiment, the present invention provides a process for the preparation of crystalline abrocitinib, the process comprising:

(a) dissolving abrocitinib in tetrahydrofuran-methanol mixture;
(b) obtaining crystalline abrocitinib from the solution of step (a) by removing the solvent from the solution obtained in (a); and
(c) isolating the crystalline abrocitinib.

In one embodiment, the present invention provides a process for the preparation of crystalline abrocitinib, the process comprising:

(a) dissolving abrocitinib in ethyl acetate-methanol mixture;
(b) obtaining crystalline abrocitinib from the solution of step (a) by removing the solvent from the solution obtained in (a); and
(c) isolating the crystalline abrocitinib.

In one embodiment, the present invention provides a process for the preparation of crystalline abrocitinib, the process comprising:

(a) dissolving abrocitinib in methylene dichloride-methanol mixture;
(b) obtaining crystalline abrocitinib from the solution of step (a) by removing the solvent from the solution obtained in (a); and
(c) isolating the crystalline abrocitinib.

In one embodiment, the present invention provides a process for the preparation of crystalline abrocitinib, the process comprising:

(a) dissolving abrocitinib in acetone-tetrahydrofuran mixture;
(b) obtaining crystalline abrocitinib from the solution of step (a) by removing the solvent from the solution obtained in (a); and
(c) isolating the crystalline abrocitinib.

In one embodiment, the present invention provides a process for the preparation of crystalline abrocitinib, the process comprising:

(a) dissolving abrocitinib in a solvent selected from the group consisting of ethers, ketones, esters, haloalkanes, amides, alcohols, and mixtures thereof;
(b) obtaining crystalline abrocitinib from the solution of step (a) by treating the solution of step (a) with an anti-solvent to form a mixture and optionally, cooling and stirring the obtained mixture; and
(c) isolating the crystalline abrocitinib.

In one embodiment, the solvent used in step (a) is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof.

In one embodiment, the solvent used in step (a) is C₁₋₈ amides and the anti-solvent used in step (b) is water.

In one embodiment, the solvent used in step (a) is C₁₋₈ amides such as dimethylformamide and the anti-solvent used in step (b) is water.

In one embodiment, the present invention provides a process for the preparation of crystalline abrocitinib, the process comprising:

(a) dissolving abrocitinib in solvent selected from C₁₋₈ amides;
(b) obtaining crystalline abrocitinib from the solution of step (a) by treating the solution of step (a) with water to form a mixture and optionally, cooling and stirring the obtained mixture; and
(c) isolating the crystalline abrocitinib.

In one embodiment, the present invention provides a process for the preparation of crystalline abrocitinib, the process comprising:

(a) dissolving abrocitinib in dimethylformamide;
(b) obtaining crystalline abrocitinib from the solution of step (a) by treating the solution of step (a) with water to form a mixture and optionally, cooling and stirring the obtained mixture; and
(c) isolating the crystalline abrocitinib.

In one embodiment, the present invention provides a process for the preparation of crystalline abrocitinib characterized by X-ray powder diffraction (XRPD) spectrum having peak reflections at about 12.9, 14.7, 19.4, 23.2 and 25.2±0.2 degrees 2 theta, the process comprising:

(a) dissolving abrocitinib in a solvent selected from the group consisting of C₂₋₁₀ ethers, C₃₋₁₀ ketones, C₂₋₁₀ esters, C₁₋₆ haloalkanes, C₁₋₈ amides, C₁₋₆ alcohols, and mixtures thereof;
(b) obtaining crystalline abrocitinib from the solution of step (a) by any of the process comprising:
(i) cooling and stirring the solution obtained in (a); or
(ii) removing the solvent from the solution obtained in (a); or
(iii) treating the solution of step (a) with an anti-solvent to form a mixture and optionally, cooling and stirring the obtained mixture; and
(c) isolating the crystalline abrocitinib.

In one embodiment, the isolated crystalline abrocitinib may be further dried. Drying may be suitably carried out in an equipment known in the art, such as a tray drier, vacuum oven, air oven, fluidized bed drier, spin flash drier, flash drier and the like. The drying may be carried out at temperatures from about room temperature to about 100° C. with or without vacuum. The drying may be carried out for any desired time until the required product quality is achieved. The drying time may vary from about 1 hour to about 25 hours, or longer.

In one embodiment, the present invention provides an amorphous solid dispersion comprising abrocitinib or salt thereof together with at least one pharmaceutically acceptable carrier.

In one embodiment, the pharmaceutically acceptable carrier is selected from a group consisting of one or more of a povidone, meglumine, gum, ethyl cellulose, hydroxypropyl cellulose, hydroxypropyl methyl cellulose, hydroxypropyl methyl cellulose-acetate succinate, hydroxypropyl methyl cellulose-phthalate, hydroxypropyl ethyl cellulose, microcrystalline cellulose, cyclodextrin, gelatin, hypromellose phthalate, lactose, polyhydric alcohol, polyethylene glycol, polyethylene oxide, polyoxyalkylene derivative, methacrylic acid copolymer, polyvinyl alcohol, polyvinyl pyrrolidone, propylene glycol derivative, fatty acid, fatty alcohols, or esters of fatty acids.

Useful pyrrolidones include, but are not limited to homopolymers or copolymers of N-vinylpyrrolidone. Such polymers can form complexes with a variety of compounds. The water-soluble forms of N-vinylpyrrolidone are available in a variety of viscosity and molecular weight grades such as but not limited to PVP K-12, PVP K-15, PVP K-17, PVP K-25, PVP K-30, PVP K-90, PVP K-120 and crospovidone.

Polyethylene glycols, condensation polymers of ethylene oxide and water, are commercially available from various manufacturers in average molecular weights ranging from about 300 to about 10,000,000 Daltons. Some of the grades that are useful in the present invention include, but are not limited to, PEG 1500, PEG 4000, PEG 6000, PEG 8000, etc.

Among various cyclodextrins α-, β-, γ- and ε-cyclodextrins or their methylated or hydroxyalkylated derivatives may be used.

In one embodiment, the pharmaceutically acceptable carrier is hydroxypropyl cellulose.

In one embodiment, the pharmaceutically acceptable carrier is polyvinyl pyrrolidone.

In one embodiment, the present invention provides an amorphous solid dispersion comprising abrocitinib with hydroxypropyl cellulose.

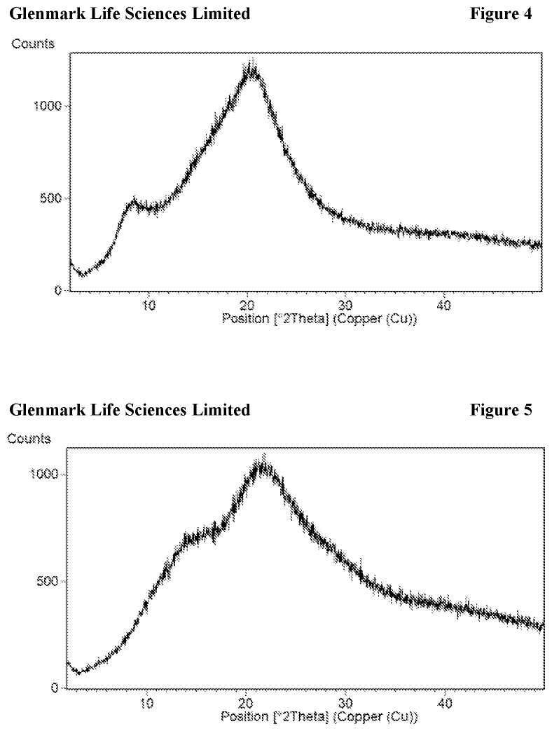

In one embodiment, the present invention provides an amorphous solid dispersion comprising abrocitinib with hydroxypropyl cellulose which is substantially in accordance with FIG. 4.

In one embodiment, the present invention provides an amorphous solid dispersion comprising abrocitinib with polyvinyl pyrrolidone.

In one embodiment, the present invention provides an amorphous solid dispersion comprising abrocitinib with polyvinyl pyrrolidone which is substantially in accordance with FIG. 5.

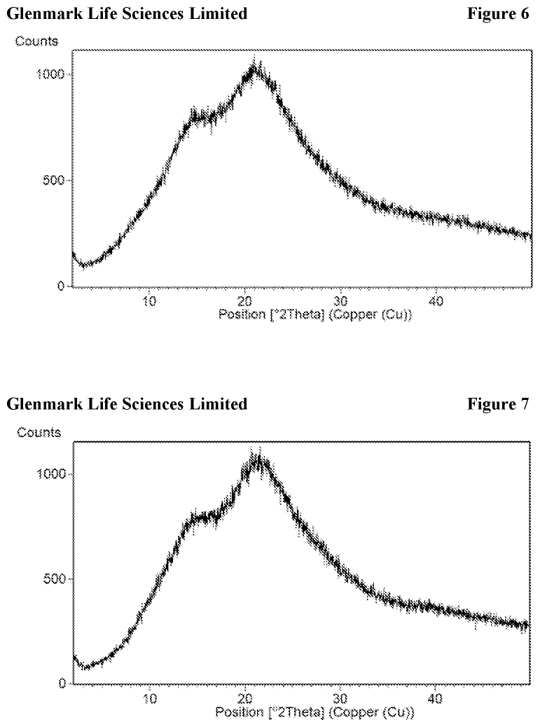

In one embodiment, the present invention provides an amorphous solid dispersion comprising abrocitinib with polyvinyl pyrrolidone which is substantially in accordance with FIG. 6.

In one embodiment, the present invention provides an amorphous solid dispersion comprising abrocitinib with polyvinyl pyrrolidone which is substantially in accordance with FIG. 7.

In one embodiment, the present invention provides a process for the preparation of an amorphous solid dispersion of abrocitinib or salt thereof together with at least one pharmaceutically acceptable carrier, the process comprising:

(a) providing a solution or mixture of abrocitinib or salt thereof together with at least one pharmaceutically acceptable carrier in a solvent; and
(b) obtaining the amorphous solid dispersion of abrocitinib or salt thereof together with at least one pharmaceutically acceptable carrier from the solution or mixture of step (a).

In one embodiment, the step (a) of providing a solution or mixture of abrocitinib or salt thereof for the preparation of amorphous solid dispersion, involves mixing with at least one pharmaceutically acceptable carrier as described herein above with a suitable solvent.

In one embodiment, the solvent is selected from the group consisting of ethers, ketones, esters, haloalkanes, amides, alcohols, water, and mixtures thereof.

In one embodiment, the solvent is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, water, and mixtures thereof.

In one embodiment, the solvent is selected from the group consisting of C₂₋₁₀ ethers such as such as diethyl ether, diisopropyl ether, methyl tert-butyl ether, tetrahydrofuran, dioxane, dimethoxy ethane, 2-methyltetrahydrofuran and the like; C₃₋₁₀ ketones such as acetone, methyl isobutyl ketone, ethyl methyl ketone and the like; C₂₋₁₀ esters such as methyl acetate, ethyl acetate, n-propyl acetate, isopropyl acetate, tert-butyl acetate and the like; C₁₋₆ haloalkanes such as methylene dichloride, ethylene dichloride, chloroform and the like; C₁₋₈ amides such as dimethyl formamide; dimethyl acetamide and the like; C₁₋₆ alcohols such as methanol, ethanol, 1-propanol, 2-propanol, 1-butanol, 2-butanol, 1-pentanol and the like; water; and mixtures thereof.

In one embodiment, the step (b) of obtaining the amorphous solid dispersion comprises:

(i) removing the solvent from the solution or mixture obtained in (a); or
(ii) treating the solution of step (a) with an anti-solvent to form a mixture and optionally, cooling and stirring the obtained mixture.

In one embodiment, the removal of solvent in (b)(i) may be carried out by solvent distillation, concentration, spray drying, fluid bed drying, lyophilization, flash drying, spin flash drying, or thin-film drying.

In one embodiment, removal of solvent in (b)(i) may be carried out by solvent distillation, preferably under vacuum.

In one embodiment, the anti-solvent used in (b)(ii) is a solvent which on addition to the solution of step (a) causes precipitation of amorphous solid dispersion of abrocitinib with at least one pharmaceutically acceptable carrier.

In one embodiment, the anti-solvent is selected from the group consisting of C₂₋₁₀ ethers, C₃₋₁₀ ketones, C₂₋₁₀ esters, C₁₋₆ haloalkanes, C₁₋₈ amides, C₁₋₆ alcohols, water, and mixtures thereof.

In one embodiment, the anti-solvent is selected from the group consisting of C₂₋₁₀ ethers such as such as diethyl ether, diisopropyl ether, methyl tert-butyl ether, tetrahydrofuran, dioxane, dimethoxy ethane, 2-methyltetrahydrofuran and the like; C₃₋₁₀ ketones such as acetone, methyl isobutyl ketone, ethyl methyl ketone and the like; C₂₋₁₀ esters such as methyl acetate, ethyl acetate, n-propyl acetate, isopropyl acetate, tert-butyl acetate and the like; C₁₋₆ haloalkanes such as methylene dichloride, ethylene dichloride, chloroform and the like; C₁₋₈ amides such as dimethyl formamide; dimethyl acetamide and the like; C₁₋₆ alcohols such as methanol, ethanol, 1-propanol, 2-propanol, 1-butanol, 2-butanol, 1-pentanol and the like; water; and mixtures thereof.

In one embodiment, the amorphous solid dispersion of abrocitinib with at least one pharmaceutically acceptable carrier prepared using the process of the present invention, contains abrocitinib in amorphous form together with at least one pharmaceutically acceptable carrier.

In one embodiment, the present invention provides an amorphous abrocitinib.

In one embodiment, the present invention provides pharmaceutical compositions comprising abrocitinib or salt thereof obtained by the processes herein described, having a D₉₀ particle size of less than about 250 microns, preferably less than about 150 microns, more preferably less than about 50 microns, still more preferably less than about 20 microns, still more preferably less than about 15 microns and most preferably less than about 10 microns.

In one embodiment, the present invention provides pharmaceutical compositions comprising abrocitinib or salt thereof obtained by the processes herein described, having a D₅₀ particle size of less than about 250 microns, preferably less than about 150 microns, more preferably less than about 50 microns, still more preferably less than about 20 microns, still more preferably less than about 15 microns and most preferably less than about 10 microns.

The particle size disclosed here can be obtained by, for example, any milling, grinding, micronizing or other particle size reduction method known in the art to bring the solid state abrocitinib or salt thereof into any of the foregoing desired particle size range.

The examples that follow are provided to enable one skilled in the art to practice the invention and are merely illustrative of the invention. The examples should not be read as limiting the scope of the invention as defined in the features and advantages.

### EXAMPLES

### Example 1: Preparation of benzyl (3-oxocyclobutyl) Carbamate

A solution of (3-oxocyclobutyl)-carboxylic acid (150 g) and triethylamine (217.5 mL) in tetrahydrofuran (2.25 L) and toluene (2.25 L) was treated with diphenyl phosphoryl azide (283.5 mL) slowly at about below 30° C. The solution warmed to about 60° C. and maintained for about 45 minutes. After 1 hour, benzyl alcohol (150 mL) was added and the solution was kept at about 60° C. for about 2 hours. After cooling to about room temperature, the solution was diluted with ethyl acetate, washed with saturated aqueous sodium bicarbonate, hydrochloric acid, sodium bicarbonate, distilled and purified by silica gel chromatography (hexane-ethyl acetate). Yield: 70 g

### Example 2: Preparation of benzyl [cis-3-(methylamino)cyclobutyl]carbamate Hydrochloride

A 2M solution of methylamine in THF (562.2 mL) was slowly added to a stirred slurry of benzyl (3-oxocyclobutyl) carbamate (60 g) and acetic acid (33 mL) at about room temperature. The mixture was stirred at about room temperature for about 2.5 hours and then cooled to about 0° C. Sodium borohydride (33.72 g) was added in portions over about 10 minutes. The mixture was warmed to about room temperature overnight. The mixture was quenched with water and concentrated under vacuum to remove tetrahydrofuran. Water was added to the mixture. The aqueous layer was acidified with concentrated hydrochloric acid to about pH 2 at about 0° C. to about 5° C., washed with ethyl acetate, basified with sodium hydroxide to about pH 9-10 and then extracted with dichloromethane. The combined organic layers were washed with brine and concentrated to obtain the product as a pale yellow liquid which was dissolved in isopropyl alcohol and cooled to about 0° C. To the resulting solution was added a solution of hydrochloric acid in isopropyl alcohol. The mixture was stirred at about 0° C. for about 30 minutes and then at about room temperature for about 12 hours. The reaction mixture was filtered, washed by isopropyl alcohol and dried at 40° C. under vacuum to give 39.2 g as crude off-white compound. The crude compound (35 g) was dissolved in isopropyl alcohol, heptane, isopropyl alcohol-hydrochloric acid solution at about 70° C. The solution was cooled to about room temperature and stirred for about 4 hours. The solid was filtered and dried under vacuum at about 40° C. to afford the cis-isomer as a white solid. Yield: 21.1 g (33%)

### Example 3: Preparation of 4-chloro-7-[(4-methylphenyl)sulfonyl]-7H-pyrrolo[2,3-d]pyrimidine

To a suspension of 2,4-dichloro-7H-pyrrolo[2,3-d]pyrimidine (50 g) and toluenesulfonyl chloride (68.2 g) in acetone (500 mL) at about 0° C. to about 5° C., a solution of sodium hydroxide in water (16.9 g in 200 mL water) was slowly added. The temperature of the mixture was raised to about room temperature and the mixture was stirred for about 3 hours. The mixture was filtered and washed with acetone-water. The solid was dried under vacuum at about 50° C. to about 55° C. for about 10 hours to give the title compound as yellow colored solid. Yield: 93.8 g (93%)

### Example 4: Preparation of benzyl [cis-3-(methyl{7-[(4-methylphenyl) sulfonyl]-7H-pyrrolo-[2,3-d]pyrimidin-4-yl}amino) cyclobutyl] carbamate

4-Chloro-7-[(4-methylphenyl)sulfonyl]-7H-pyrrolo[2,3-d]pyrimidine (26 g) and benzyl [cis-3-(methylamino)cyclobutyl]carbamate hydrochloride (30 g) were mixed with isopropyl alcohol (312 mL) and diisopropylethyl amine (42.26 mL). The slurry was heated at about 75° C. for about 6 hours. The mixture was cooled to about room temperature, filtered, washed with isopropyl alcohol and dried at about 40° C. under vacuum to give the title compound as a white solid. Yield: 40.1 g (72%)

### Example 5: Preparation of cis-N-methyl-N-{7-[(4-methylphenyl)sulfonyl]-7H-pyrrolo[2,3-d]-pyrimidin-4-yl} cyclobutane-1,3-diamine Dihydrobromide

Benzyl [cis-3-(methyl {7-[(4-methylphenyl)sulfonyl]-7H-pyrrolo-[2,3-d]pyrimidin-4-yl}amino) cyclobutyl] carbamate (35 g) was suspended in ethyl acetate (105 mL) and acetic acid (105 mL). A 4M solution of hydrobromic acid in acetic acid (105 mL) was slowly added to the mixture and the temperature of the mixture was maintained about below 25° C. The mixture was stirred at about room temperature for about 2 hours. The solid obtained was filtered, washed with ethyl acetate and dried at about 40° C. under vacuum to give the title compound as a white solid. Yield: 37 g (95%)

### Example 6: Preparation of Abrocitinib

Cis-N-methyl-N-{7-[(4-m ethylphenyl)sulfonyl]-7H-pyrrolo[2,3-d]-pyrimidin-4-yl} cyclobutane-1,3-diamine dihydrobromide (32 g) was added in portions to a mixture of 2-methyltetrahydrofuran (320 mL) and triethylamine (113.15 mL). The mixture was stirred at about room temperature for about 1 hour and 1-propanesulfonyl chloride (11.52 mL) was added over about 10 min. The mixture was stirred for about 1 hour at about room temperature. The mixture was washed with 10% aqueous citric acid solution. Aqueous 3M sodium hydroxide solution was added to the mixture and the mixture was heated to reflux with stirring for about 1 hour. The mixture was cooled to about room temperature and the layers were separated. The organic layer was extracted with aqueous sodium hydroxide. The aqueous layers were combined, cooled to about 15° C. and acidified to about pH 6 by slow addition of dilute hydrochloric acid solution. The mixture was cooled to about 5° C. and stirred for about 1 hour at about the same temperature. The tan granular solid was filtered, washed with water and dried at about 45° C. under vacuum. Yield: 11.1 g (57%).

### Example 7: Preparation of Abrocitinib

A mixture of abrocitinib (500 mg) in ethanol (3.34 mL) and water (1.67 mL) was heated to reflux until all solids dissolved. The solution to slowly cooled to about room temperature. The solid obtained was filtered, washed with ethanol-water and dried under vacuum at about 40° C. Yield: 374 mg (75%)

### Example 8: Preparation of Abrocitinib

To a solution of abrocitinib (500 mg) and tetrahydrofuran (4 mL) was dissolved by adding methanol (3 mL) dropwise at about reflux temperature. The solution was cooled slowly to about room temperature and stirred for about 1-2 hours. The solid obtained was filtered and dried under vacuum at about 40° C. Yield: 342 mg (68%)

TABLE 1
XRD peaks of abrocitinib

| Pos. | d-spacing | Rel. Int. |
|---|---|---|
| [°2Th.] | [Å] | [%] |
| 8.83 | 10.00 | 1.61 |
| 10.80 | 8.18 | 1.37 |
| 12.62 | 7.01 | 19.42 |
| 13.03 | 6.79 | 30.52 |
| 13.45 | 6.58 | 2.63 |
| 14.86 | 5.95 | 100.00 |
| 15.84 | 5.59 | 4.24 |
| 16.62 | 5.33 | 5.31 |
| 17.63 | 5.02 | 22.63 |
| 18.08 | 4.90 | 41.84 |
| 18.65 | 4.75 | 4.45 |
| 19.53 | 4.54 | 42.62 |
| 20.32 | 4.36 | 13.46 |
| 20.69 | 4.29 | 15.65 |
| 21.74 | 4.08 | 4.71 |
| 22.31 | 3.98 | 2.05 |
| 23.33 | 3.81 | 76.75 |
| 24.32 | 3.65 | 6.48 |
| 24.78 | 3.59 | 9.91 |
| 25.01 | 3.55 | 22.34 |
| 25.39 | 3.50 | 33.20 |
| 25.98 | 3.42 | 9.63 |
| 26.51 | 3.36 | 4.86 |
| 26.88 | 3.31 | 11.23 |
| 27.35 | 3.26 | 7.52 |
| 28.68 | 3.11 | 1.83 |
| 29.94 | 2.98 | 1.80 |
| 30.42 | 2.93 | 3.49 |
| 31.80 | 2.81 | 15.59 |
| 32.62 | 2.74 | 3.01 |
| 34.09 | 2.62 | 1.17 |
| 35.38 | 2.53 | 3.29 |
| 36.47 | 2.46 | 2.07 |
| 37.59 | 2.39 | 5.22 |
| 38.31 | 2.34 | 1.92 |
| 39.53 | 2.27 | 2.41 |
| 40.05 | 2.25 | 4.07 |
| 40.84 | 2.20 | 2.55 |

DSC (Exo): 189.73° C.

### Example 9: Preparation of Abrocitinib

To a solution of abrocitinib (500 mg) and acetone (5 mL) was dissolved by adding methanol (˜7.0 mL) dropwise at about reflux temperature. The solution was cooled slowly to about room temperature and stirred for about 4-5 hours. The solid obtained was filtered and dried under vacuum at about 40° C. Yield: 281 mg (56%); XRD: 12.9, 14.7, 19.4, 23.2, 25.2±0.2 degrees 2 theta; DSC (Exo): 190.64° C.

### Example 10: Preparation of Abrocitinib

To a solution of abrocitinib (500 mg) and ethyl acetate (5 mL) was dissolved by adding methanol (˜7.5 mL) dropwise at about reflux temperature. The solution was cooled slowly to about room temperature and stirred for about 4-5 hours. The solid obtained was filtered and dried under vacuum at about 40° C. Yield: 276 mg (55%); XRD: 12.9, 14.7, 19.4, 23.2, 25.2±0.2 degrees 2 theta; DSC (Exo): 189.89° C.

### Example 11: Preparation of Abrocitinib

To a solution of abrocitinib (500 mg) and tetrahydrofuran (20 mL) was added methanol (20 mL). The mixture was heated and stirred at about reflux temperature to give a clear solution. The solvent was evaporated completely under reduced pressure at about below 50° C. Yield: 482 mg (96%); XRD: 12.9, 14.7, 19.4, 23.2, 25.2±0.2 degrees 2 theta; DSC (Exo): 189.22° C.

### Example 12: Preparation of Abrocitinib

To a solution of abrocitinib (500 mg) and ethyl acetate (15 mL) was added methanol (15 mL). The mixture was heated and stirred at reflux temperature to give a clear solution. The solvent was evaporated completely under reduced pressure at about below 50° C. Yield: 479 mg (96%); XRD: 12.9, 14.7, 19.4, 23.2, 25.2±0.2 degrees 2 theta; DSC (Exo): 189.0° C.

### Example 13: Preparation of Abrocitinib

To a solution of abrocitinib (500 mg) and dichloromethane (15 mL) was added methanol (15 mL). The mixture was stirred at about room temperature to give a clear solution. The solvent was evaporated completely under reduced pressure at about below 40° C. Yield: 481 mg (96%); XRD: 12.9, 14.7, 19.4, 23.2, 25.2±0.2 degrees 2 theta; DSC (Exo): 189.61° C.

### Example 14: Preparation of Abrocitinib

To a mixture of abrocitinib (500 mg) and acetone (15 mL) was added tetrahydrofuran (15 mL) at about room temperature. The mixture was heated and stirred at about reflux temperature to give a clear solution. The solvent was evaporated completely under reduced pressure at about below 40° C. Yield: 482 mg (96%); XRD: 12.9, 14.7, 19.4, 23.2, 25.2±0.2 degrees 2 theta; DSC (Exo): 189.84° C.

### Example 15: Preparation of Abrocitinib

To a solution of abrocitinib (500 mg) and dimethylformamide (5 mL) was added water (15 mL) dropwise at about room temperature. The precipitated solid was stirred for about 2 hours at about room temperature. The solid obtained was filtered and dried at about 60° C. Yield: 413 mg (82%); XRD: 12.9, 14.7, 19.4, 23.2, 25.2±0.2 degrees 2 theta; DSC (Exo): 190.85° C.

### Example 16: Preparation of Amorphous Solid Dispersion of Abrocitinib with Hydroxypropyl Cellulose (HPC)

Abrocitinib (0.25 g) and hydroxypropyl cellulose (0.75 g) were dissolved in methanol:acetone (1:1 v/v) solvent mixture (25 mL). The solution was stirred at about room temperature for about 15-20 minutes. The solution was filtered for particle free solution. The obtained clear solution was distilled off under high vacuum at about 40° C. to about 45° C. using rotavapour. The obtained solid was dried for about 30 minutes at about 40° C. to about 45° C.

### Example 17: Preparation of Amorphous Solid Dispersion of Abrocitinib with Polyvinyl Pyrrolidone (PVP K90)

Abrocitinib (0.25 g) and polyvinyl pyrrolidone K90 (0.75 g) were dissolved in methanol:acetone (1:1) solvent mixture (20 mL). The solution was stirred at about room temperature for about 15-20 minutes. The solution was filtered for particle free solution. The obtained clear solution was distilled off under high vacuum at about 40° C. to about 45° C. using rotavapour. The obtained solid was dried for about 30 minutes at about 40° C. to about 45° C.

### Example 18: Preparation of Amorphous Solid Dispersion of Abrocitinib with Polyvinyl Pyrrolidone (PVP K90)

Abrocitinib (0.25 g) and polyvinyl pyrrolidone K90 (0.25 g) was dissolved in methanol:acetone (1:1) solvent mixture (20 mL). The solution was stirred at about room temperature for about 15-20 minutes. The solution was filtered for particle free solution. The obtained clear solution was distilled off under high vacuum at about 40° C. to about 45° C. using rotavapour. The obtained solid was dried for about 30 minutes. at about 40° C. to about 45° C.

### Example 19: Preparation of Amorphous Solid Dispersion of Abrocitinib with Polyvinyl Pyrrolidone (PVP K90)

Abrocitinib (0.25 g) and polyvinyl pyrrolidone K90 (0.5 g) was dissolved in methanol:acetone (1:1) solvent mixture (20 mL). The solution was stirred at about room temperature for about 15-20 minutes. The solution was filtered for particle free solution. The obtained clear solution was distilled off under high vacuum at about 40° C. to about 45° C. using rotavapour. The obtained solid was dried for about 30 minutes at about 40° C. to about 45° C.

1. A process for the preparation of crystalline abrocitinib characterized by X-ray powder diffraction (XRPD) spectrum having peak reflections at about 12.9, 14.7, 19.4, 23.2 and 25.2±0.2 degrees 2 theta, the process comprising:
(a) dissolving abrocitinib in a solvent selected from the group consisting of ethers, ketones, esters, haloalkanes, amides, alcohols, and mixtures thereof;
(b) obtaining crystalline abrocitinib from the solution of step (a); and
(c) isolating the crystalline abrocitinib.

2. The process of claim 1, wherein abrocitinib is dissolved at a temperature range from 20° C. to reflux temperature of the solvent.

3. The process of claim 1, wherein the step (b) of obtaining crystalline abrocitinib comprises:
(i) cooling and stirring the solution obtained in step (a); or
(ii) removing the solvent from the solution obtained in step (a); or
(iii) treating the solution of step (a) with an anti-solvent to form a mixture and optionally, cooling and stirring the obtained mixture.

4. The process of claim 3, wherein the step (b)(i) of cooling is carried out at a temperature of 0° C. to 30° C.

5. The process of claim 3, wherein the anti-solvent is selected from the group consisting of hydrocarbons, ethers, ketones, esters, haloalkanes, amides, alcohols, water, and mixtures thereof.

6. An amorphous solid dispersion comprising abrocitinib or salt thereof together with at least one pharmaceutically acceptable carrier.

7. The amorphous solid dispersion of claim 6, wherein the at least one pharmaceutically acceptable carrier is selected from a group consisting of a povidone, meglumine, gum, ethyl cellulose, hydroxypropyl cellulose, hydroxypropyl methyl cellulose, hydroxypropyl methyl cellulose-acetate succinate, hydroxypropyl methyl cellulose-phthalate, hydroxypropyl ethyl cellulose, microcrystalline cellulose, cyclodextrin, gelatin, hypromellose phthalate, lactose, polyhydric alcohol, polyethylene glycol, polyethylene oxide, polyoxyalkylene derivative, methacrylic acid copolymer, polyvinyl alcohol, polyvinyl pyrrolidone, propylene glycol derivative, fatty acid, fatty alcohols, Of esters of fatty acids and mixtures thereof.

8. The amorphous solid dispersion of claim 6, wherein the at least one pharmaceutically acceptable carrier is hydroxypropyl cellulose.

9. The amorphous solid dispersion of claim 6, wherein the at least one pharmaceutically acceptable carrier is polyvinyl pyrrolidone.

10. A process for the preparation of an amorphous solid dispersion of abrocitinib or salt thereof together with at least one pharmaceutically acceptable carrier, the process comprising:
(a) providing a solution or mixture of abrocitinib or salt thereof together with at least one pharmaceutically acceptable carrier in a solvent; and
(b) obtaining the amorphous solid dispersion of abrocitinib or salt thereof together with the at least one pharmaceutically acceptable carrier from the solution or mixture of step (a).

11. The process of claim 10, wherein the solvent is selected from the group consisting of ethers, ketones, esters, haloalkanes, amides, alcohols, water, and mixtures thereof.

12. The process of claim 10, wherein the step (b) of obtaining the amorphous solid dispersion comprises:
(i) removing the solvent from the solution or mixture obtained in step (a); or
(ii) treating the solution of step (a) with an anti-solvent to form a mixture and optionally, cooling and stirring the obtained mixture.

13. The process of claim 12, wherein the anti-solvent is selected from the group consisting of hydrocarbons, ethers, ketones, esters, haloalkanes, amides, alcohols, water, and mixtures thereof.

14. The process of claim 10, wherein the at least one pharmaceutically acceptable carrier is selected from a group consisting of a povidone, meglumine, gum, ethyl cellulose, hydroxypropyl cellulose, hydroxypropyl methyl cellulose, hydroxypropyl methyl cellulose-acetate succinate, hydroxypropyl methyl cellulose-phthalate, hydroxypropyl ethyl cellulose, microcrystalline cellulose, cyclodextrin, gelatin, hypromellose phthalate, lactose, polyhydric alcohol, polyethylene glycol, polyethylene oxide, polyoxyalkylene derivative, methacrylic acid copolymer, polyvinyl alcohol, polyvinyl pyrrolidone, propylene glycol derivative, fatty acid, fatty alcohols, esters of fatty acids and mixtures thereof.

15. The process of claim 10, wherein the at least one pharmaceutically acceptable carrier is hydroxypropyl cellulose.

16. The process of claim 10, wherein the at least one pharmaceutically acceptable carrier is polyvinyl pyrrolidone.
